(19) United States
(12) Patent Application Publication
(10) Pub. No.: US 2025/0195542 A1
(43) Pub. Date: Jun. 19, 2025

(54) METABOLIC DISEASE THERAPEUTIC AGENT OR PREVENTIVE AGENT

(71) Applicant: Peking University, Beijing (CN)

(72) Inventors: Min YE, Beijing (CN); Yi KUANG, Beijing (CN); Xue QIAO, Beijing (CN); Huifei SU, Beijing (CN)

(73) Assignee: Peking University, Beijing (CN)

(21) Appl. No.: 18/847,925

(22) PCT Filed: Mar. 17, 2022
(86) PCT No.: PCT/CN2022/081436

§ 371 Date: Sep. 17, 2024

(51) Int. Cl. A61K 31/575; A61K 31/704; A61K 36/07; A61P 1/16

(52) U.S. Cl. CPC A61K 31/575; A61K 31/704; A61K 36/07; A61P 1/16; A61K 2236/15; A61K 2236/33

(57) ABSTRACT

The present invention relates to a use of a compound and an extract derived from natural fungus Antrodia camphorata in the preparation of an FGF21 agonist and/or an RDH10 agonist.

### TECHNICAL FIELD

The present invention relates to the use of compounds and extracts derived from natural fungus Antrodia camphorata for the preparation of FGF21 agonists and/or RDH10 agonists, and therapeutic or preventive agents for non-alcoholic steatohepatitis and other related diseases.

### BACKGROUND TECHNOLOGY

Fibroblast growth factor 21 (FGF21) is a metabolism related protein. It is a member of the fibroblast growth factor (FGF) family and has the function of endocrine factors. Its biological activities include promoting cell proliferation, body development, vascular proliferation, and wound repair, participating in the body's substance metabolism, and maintaining the balance of body lipid and glucose metabolism. FGF21 is highly expressed in pancreas, liver and white adipose tissue, and is mainly regulated by PPAR and insulin/akt1 pathways. Under the regulation of PPARγ in liver and adipose tissue, FGF21 modulates lipid metabolism through the interaction between cell surface receptor FGFR1c and co-receptor β-Klotho. (Fisher F M et al, Annu Rev Physiol. 2016; 78: 223-241) The studies have showed that the upregulation of FGF21 may be a protective mechanism and compensatory response against lipid toxicity. (Bonakdaran S et al., Acta Endocrinol-Buch. 2017; 13: 278-281).

As a metabolic regulator, FGF21 is of great value in the treatment of metabolic diseases such as lipid metabolism disorders. (Tezze C et al, Front Physiol. 2019; 10: 419) It may become a new therapeutic target for the treatment of metabolic diseases. Moreover, FGF21 is the only protein found in FGF family that does not promote mitosis, which greatly reduces the risk of clinical use.

In addition, studies have shown that endoplasmic reticulum stress induces an increase in FGF21 expression and synthesis in the liver, and administration of exogenous FGF21 inhibits hepatic steatosis induced by endoplasmic reticulum stress, suggesting that FGF21 can ameliorate endoplasmic reticulum stress, which in turn may reduce the damage caused by non-alcoholic steatohepatitis (NAFLD). (Inagaki T et al., Front Endocrinol. 2015; 6: 147)

At present, most of the drugs acting on the new target FGF21 in clinic are FGF21 analogues, such as LY2405319, PF-05231023, BMS-986036 and other macromolecules or polypeptides, are used to improve lipid metabolism disorders. For example, FGF21 analogue BMS-986036 has been found that can be used as an FGF21 agonist. However, the effective doses of the above FGF21 analogues are too high, which may be related to FGF21 resistance in patients with metabolic diseases. (Gaich G et al, Cell Metab. 2013; 18: 333-340) In addition, as polypeptides, the above FGF21 analogues need to be administered by injection and cannot be administered orally. Due to the high renal clearance rate and proteolytic cleavage, the half-life of blood circulation is low (0.5-2 h) and the pharmacokinetic properties are poor. (Lee J H et al, Am J Transl Res. 2016; 8:4750-4763) Preclinical studies have also found that FGF21 analogues can cause a large amount of bone loss, and lead to osteoporosis. (Wei W et al, Proc Natl Acad Sci USA. 2012; 109: 3143-3148) FGF21 analogues can also inhibit liver growth hormone insulin-like growth factor axis and affect body growth, which may not be suitable for children and adolescents. (Inagaki T et al, Cell Metab. 2008; 8: 77-83)

In addition, no small molecule agonist of FGF21 with favorable effects has been found. There are only a few studies on drugs such as obeticholic acid, metformin and talabostat. Among them, it is reported that obeticholic acid can upregulate FGF21. (Hu Y et al, JHEP reports. 2020; 2: 100093) However, its upregulation effect is relatively weak, and it shows serious side effects in clinical trials, including severe pruritus.

Another metabolism related protein is retinol dehydrogenase 10 (RDH10). RDH10 is a subtype of retinol dehydrogenase. It belongs to the short chain dehydrogenase/reductase (SDR) superfamily and consists of 341 amino acids. It is expressed in retina, kidney, liver, small intestine, placenta, lung, heart, skeletal muscle and other tissues and organs. RDH10 has been proved to have the highest affinity for retinol among SDR enzymes, catalyzing the oxidation of all-trans and 11-cis retinol to produce the relevant retinoaldehyde. This reaction is the first step of the metabolism that turns retinol to retinoic acid. (Belyaeva O V, et al., J Biol Chem 283, 29, 20299-20308) RDH10 also plays an important role in the metabolic homeostasis of all-trans retinoic acid. It is reported that the level of all-trans retinoic acid synthesis in RDH10 heterozygous mice is decreased, which leads to lipid deposition. (Yang D, et al., Diabetes 2018, 67, 662-673) These results indicate that RDH10 or retinoic acid is related to lipid deposition.

Therefore, in order to treat diseases that can benefit from the modulation of FGF21 and/or RDH10, it is still necessary to find more effective FGF21 agonists and/or RDH10 agonists. In particular, substances that have one or both of the FGF21 agonistic and RDH10 agonistic effects may become better therapeutic or preventive agents for lipid metabolism disorders and diseases associated with lipid metabolism abnormalities.

The inventors of this invention unexpectedly discover that extracts and specific chemical components from natural fungus Antrodia camphorata exhibit FGF21 agonistic and/or RDH10 agonistic effects. Antrodia camphorata, also known as Cinnamomum camphora mushroom, is a species unique to Taiwan, growing exclusively in the decayed heartwood of Cinnamomum kanehirai trees at altitudes of 450 to 2000 meters in the mountains or on the dark, damp surfaces of the fallen, decayed wood. The mushroom is a very precious resource, and local people use it as a traditional medicine to treat abdominal pain, allergies, hangovers, and liver injury. To date, it has no report about Antrodia camphorata or any of its components being used as agonists for the FGF family, especially FGF21, or for the RDH family, especially RDH10. Additionally, although DEA, a component in the ethanol extract of Antrodia camphorata, is reported to prevent and treat non-alcoholic steatohepatitis, its effect is relatively weak, and there is no mention whether it has FGF21 agonistic or RDH10 agonistic effects. Thus, DEA is insufficient to meet the relevant clinical needs.

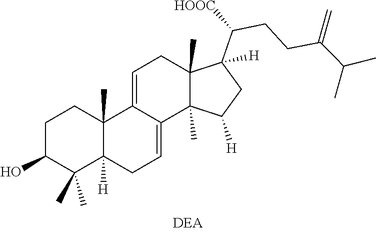

### BRIEF SUMMARY OF THE INVENTION

The inventors of this application have discovered that certain chemical components, their derivatives, and specific extracts containing these components from Antrodia camphorata have FGF21 agonistic and/or RDH10 agonistic effects. These substances can significantly prevent or reduce lipid deposition and can be used for the prevention and treatment of lipid metabolism disorders or/and diseases associated with lipid metabolism abnormalities, thereby completing the present invention.

Therefore, the present invention includes, but is not limited to, the following contents:

One aspect of the present invention relates to the use of a compound of formula (I), or its salts or isomers, in the preparation of FGF21 agonists and/or RDH10 agonists.

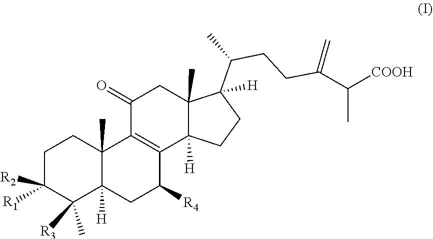

Wherein R₁ and R₂ are each independently selected from the group consisting of —H, —OH and C₁₋₆ alkyl, or R₁ and R₂ together form ═O; R₃ is selected from the group consisting of —H, —OH and C₁₋₆ alkyl; and R₄ is selected from the group consisting of —H and —OH, or glycosyl (preferably oxy-glucosyl).

In a preferred embodiment, R₁ and R₂ are each independently selected from the group consisting of —OH and —H, or R₁ and R₂ together form ═O; R₃ is selected from the group consisting of —H and —OH; and R₄ is —OH or oxy-glucose.

In a further preferred embodiment, the compound of formula (I) is Antcin K, Antcin C, AK-GLU (Antcin K-7-O-glucoside), AC-GLU (Antcin C-7-O-glucoside), or a salt or isomer thereof:

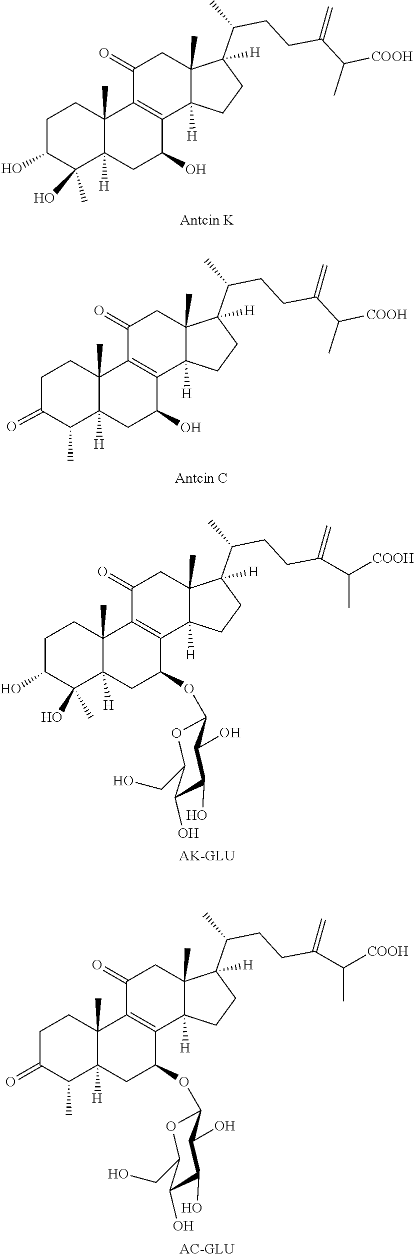

In a further preferred embodiment, the compound of formula (I) is 25S-Antcin C (the 25S epimer of Antcin C) or a salt:

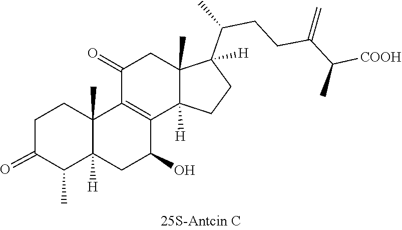 [SMILES: C=C(CC[C@@H](C)[C@H]1CC[C@H]2C3=C(C(=O)C[C@]12C)[C@@]1(C)CCC(=O)[C@@H](C)[C@@H]1C[C@@H]3O)[C@H](C)C(=O)O]

Another aspect of the present invention relates to an extract of Antrodia camphorata, which meets one or more of the following conditions:
(1) Containing more than 5% by mass of Antcin K, preferably more than 10%, and more preferably more than 15%;
(2) Containing more than 3% by mass of Antcin C, preferably more than 5%, and more preferably more than 8%;
(3) Containing more than 8% by mass of Antcin K and Antcin C in total, preferably more than 15%, and more preferably more than 20%;
(4) Containing less than 1% by mass of DEA, preferably less than 0.5%, and more preferably less than 0.1%.

Another aspect of the present invention relates to a method for extracting the aforementioned Antrodia camphorata extract, which comprises the following steps:
(1) Crush Antrodia camphorata; and
(2) Extract with a methanol-water solution (e.g., by reflux or ultrasonic extraction), wherein the methanol concentration in the solution is 20-80%, preferably 30-70%, and more preferably 40-60% (e.g., about 50%).

Another aspect of the present invention relates to a pharmaceutical composition containing the aforementioned compound or extract.

Another aspect of the present invention relates to the use of the aforementioned extract or pharmaceutical composition in the preparation of FGF21 agonists and/or RDH10 agonists.

Another aspect of the present invention relates to the use of any of the aforementioned compounds or their salts or isomers, extracts, or pharmaceutical compositions in the preparation of a drug for the treatment or prevention of lipid metabolism disorders or/and diseases associated with lipid metabolism abnormalities. Examples of lipid metabolism disorders include: hyperlipidemia; cholesterol deposition; retinal lipemia; steatohepatitis, such as non-alcoholic steatohepatitis. Diseases associated with lipid metabolism abnormalities include: obesity; cardiovascular diseases related to lipid metabolism abnormalities, such as hypertension and atherosclerosis; or/and kidney diseases associated with lipid metabolism abnormalities. Preferably, the drug is used for the treatment or prevention of non-alcoholic steatohepatitis.

The advantage of the present invention is providing the use of the aforementioned compound of formula (I), particularly Antcin K, Antcin C, AK-GLU, AC-GLU, and 25S-Antcin C, in the preparation of FGF21 agonists or therapeutic or preventive agents for related diseases. The FGF21 agonistic activity of these compounds is significantly superior to that of the known FGF21 agonist obeticholic acid in the prior art and DEA from Antrodia camphorata reported in the literature. Additionally, these compounds also exhibit significant RDH10 agonistic activity, resulting in a very notable improvement in the therapeutic effects for the aforementioned diseases, particularly non-alcoholic steatohepatitis. The invention also provides an Antrodia camphorata extract that can enrich these compounds, with a simple and efficient extraction method. The enriched extract can be directly used to prepare FGF21 agonists and/or RDH10 agonists, or therapeutic or preventive agents for the aforementioned diseases (particularly non-alcoholic steatohepatitis), thereby greatly reducing the cost of the medication.

### BRIEF DESCRIPTION OF THE DRAWINGS

FIG. 1: ¹H-NMR spectrum of Antcin K (pyridine-d₅, 400 MHz).

FIG. 2: ¹³C-NMR spectrum of Antcin K (pyridine-d₅, 100 MHz).

FIG. 3: ¹H-NMR spectrum of Antcin C (pyridine-d₅, 400 MHz).

FIG. 4: ¹³C-NMR spectrum of Antcin C (pyridine-d₅, 100 MHz).

FIG. 5: HPLC chromatograms of different extracts and standards of Antrodia camphorata in Example 3.

FIG. 6: Experimental results showing the effect on liver FGF21 gene expression in methionine-choline deficient diet-fed mice (MCD mice) in Example 4.

FIG. 7: Experimental results showing the effect on liver FGF21 protein expression in MCD mice in Example 5.

FIG. 8: Serum indicators of liver function in MCD mice in Example 6.

FIG. 9: Results of pathological staining of liver sections from MCD mice in Example 6.

FIG. 10: Experimental results showing the effect on serum FGF21 protein expression in MCD mice in Example 7.

FIG. 11: Synthesis route of 25S-Antcin C—CO—NH-PEG-biotin molecular probe in Example 8.

FIG. 12: ¹H NMR spectrum of 25S-Antcin C—CO—NH-PEG-biotin in Example 8 (pyridine-d₅, 400 MHz).

FIG. 13: ¹³C NMR spectrum of 25S-Antcin C—CO—NH-PEG-biotin in Example 8 (pyridine-d₅, 100 MHz).

FIG. 14: Results of protein pull-down assays using the molecular probe in Example 9.

FIG. 15: Experimental data on the direct target RDH10 of 25S-Antcin C in Example 10 (Note: In Figure B, arrows from top to bottom indicate concentrations corresponding to the curves from top to bottom).

FIG. 16: RDH10 siRNA cell knockdown experimental results in Example 11.

FIG. 17: Protective effects of 25S-Antcin C on the liver of MCD mice in Example 12.

### DETAILED DESCRIPTION OF EMBODIMENTS

The term “approximately” used in the context of this description means a range that fluctuates 10% above and below the cited value. For example, if the concentration of a component is about 5 mM, it means that its concentration is 4.5 to 5.5 mM; and if the concentration of a component is in the range of about 5 to 10 mM, it means that its concentration is in the range of 4.5 to 11 mM. Other terms used in this description have meanings commonly understood in the field.

The term “FGF21 agonist and/or RDH10 agonist” as described in this description means that the relevant substance may be an FGF21 agonist, an RDH10 agonist, or a dual agonist of both FGF21 and RDH10. The term “FGF21 agonistic effect and/or RDH10 agonistic effect” means that the relevant substance may exhibit FGF21 agonistic effect, an RDH10 agonistic effect, or a dual agonistic effect on both FGF21 and RDH10.

The term “compound of formula (I)” and the specific compounds Antcin K, Antcin C, AC-GLU, and AK-GLU described in this specification include their salts and isomers. The salts are preferably pharmaceutically acceptable salts formed with acids or bases. The term “isomers” includes tautomeric isomers, cis-trans isomers, non-enantiomeric isomers (epimers), and mixtures thereof, as known to those skilled in the art. In particular, the carbon atom at position 25 (i.e., the carbon atom to which the carboxyl group is attached) in the general structure and specific compounds of the invention is a chiral carbon, so the term “isomers” includes the 25R/25S epimers and any mixtures thereof (including a 1:1 ratio). Additionally, for the R and S configurations of the epimers, they may be represented as “25R” and “25S”, respectively, in this description. For example, “25R-Antcin K” and “25S-Antcin K” for Antcin K with the C-25 in the R and S configurations, respectively, and “25R-Antcin C” and “25S-Antcin C” for Antcin C with the C-25 in the R and S configurations, respectively.

In the context of this description, the term “glycosyl” refers to the group derived from the sugar portion of a glycoside formed when a glycoside compound is combined with sugar. Preferably, the “glycosyl” described in this specification is oxy-glucosyl, and its structure is shown as follows:

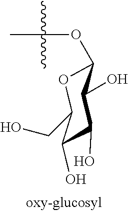

Recognizing that Antcin K and Antcin C (especially 25S-Antcin C), which are naturally occurring components in Antrodia camphorata, have significant FGF21 agonistic and/or RDH10 agonistic effects, particularly in the treatment or prevention of non-alcoholic steatohepatitis (NASH), the applicant designed a method as described earlier for extracting Antrodia camphorata using a methanol-water solution. This method can efficiently and easily obtain an extract enriched with Antcin K and Antcin C, while essentially free of impurities like DEA, as described above in the invention. For example, under the experimental conditions of Example 4, the extract of Antrodia camphorata in the invention, administered at approximately 80 mg/kg, can increase the FGF21 gene expression level in the liver of MCD mice to approximately 150 times or 200 times than that of normal mice. Under the experimental conditions of Example 5, the extract of Antrodia camphorata of the invention, administered at approximately 80 mg/kg, can increase the FGF21 protein expression level in the liver of MCD mice to approximately 100% or 120% than that of normal mice. Under the experimental conditions of Example 6, the extract of Antrodia camphorata in the invention, administered at approximately 80 mg/kg, can reduce the serum alanine aminotransferase (ALT) level in MCD mice to 120 IU/L or less, or 100 IU/L or less. Under the experimental conditions of Example 7, the extract of Antrodia camphorata in the invention, administered at approximately 80 mg/kg, can increase the serum FGF21 level in MCD mice to 200 pg/ml or more, or 300 pg/ml or more.

The compounds or extracts of the present invention can be administered to the treatment subjects, such as human patients, using any convenient means that can achieve the desired results. For example, the compounds can be formulated into pharmaceutical compositions as described earlier, and/or into known or newly developed dosage forms (such as tablets, capsules, injections, etc.). The compounds or extracts of the present invention can be used in combination with other therapeutic drugs. When used in combination, the compounds or extracts of the present invention can be formulated into the same dosage form as the other therapeutic drugs or into separate dosage forms. The pharmaceutical compositions or dosage forms mentioned above may include one or more pharmaceutically acceptable carriers or excipients, including but not limited to diluents, fillers, binders, wetting agents, disintegrants, lubricants, and so on.

### EXAMPLES

The specific embodiments of the present invention will be illustrated through the following examples. However, it should be understood that these examples are not intended to limit the use of the invention. The materials, reagents, and other substances used in the examples are well-known to those skilled in the field and can be obtained through commercial sources or literature methods. The experimental or characterization methods used are also well-known methods in the field.

### Example 1: Extraction and Separation of Antcin K and Antcin C

(R)- and (S)-1-(9-Anthryl)-2,2,2-trifluoroethanol (Sigma-Aldrich, USA), Et3N (triethylamine, J&K Scientific, Beijing), DMAP (4-(dimethylamino) pyridine, J&K Scientific, Beijing), EDCI (1-ethyl-3-(dimethylaminopropyl) carbodiimide hydrochloride, Bidepharm, Shanghai), and other chemical reagents were all purchased from Beijing Chemical Works.

First, weigh 25 kg of dried dish-cultured Antrodia camphorata and crush it. Add 10 times the volume of 95% ethanol and heat under reflux for 2-3 hours, then filter. Repeat the extraction of the residue with 95% ethanol 5 times. Combine the extract solutions, concentrate under reduced pressure to evaporate the solvent, and yield the total extract, which is the ethanol extract of Antrodia camphorata. The total extract obtained is approximately 4.8 kg, with a yield of 19.2%.

To isolate Antcin K and Antcin C from the ethanol extract, dissolve 1.2 kg of the ethanol extract in 50% ethanol. Apply it in four portions to a 9.6 kg macroporous adsorption resin column (AB-8). Use ethanol-water mixtures (50:50, 70:30, 85:15, 95:5) as the mobile phase in a gradient elution. Based on TLC and HPLC analysis results, combine the elution fractions into six fractions (A-F).

Fraction B (80.5 g) was separated using a silica gel column, with dichloromethane-methanol (15:1 to 1:1, v/v) as the mobile phase in a gradient elution. This yielded four fractions (BA-BD) and the compound Antcin K (25R S, 10 g). Further purification was performed by semi-preparative liquid chromatography (acetonitrile-water, 25:75, v/v), resulting in the isolation of 25S-Antcin K (500 mg) and 25R-Antcin K (400 mg).

Fraction D (90.5 g) was dissolved in an appropriate amount of methanol, sonicated, and filtered, yielding filtrate DA (12.6 g) and solid DB (70.8 g). Filtrate DA was separated using a silica gel column, with dichloromethane-methanol (10:1 to 1:1, v/v) as the eluent. Based on TLC and HPLC results, it was combined into four fractions, DAA to DAD. Fraction DAB (2.6 g) was further separated using LH-20 gel chromatography to obtain two fractions (DABA and DABB), and fraction DABA (309.1 mg) was subjected to semi-preparative liquid chromatography (acetonitrile-water, 65:36, v/v), resulting in the isolation of 25R-Antcin C (50.3 mg) and 25S-Antcin C (60.4 mg).

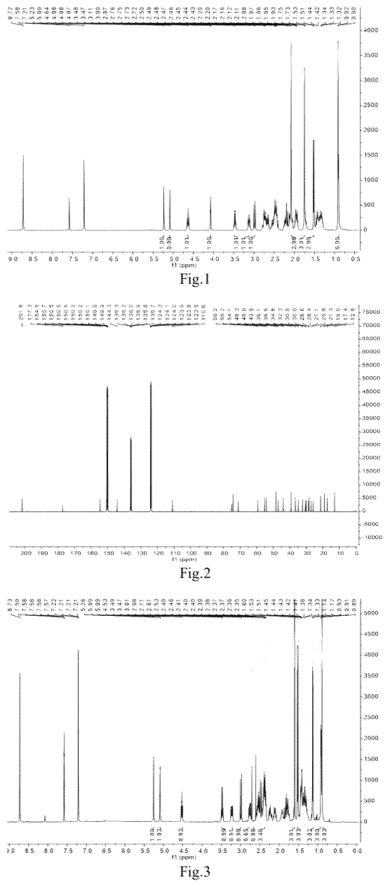

### Structural Identification (See FIGS. 1 to 4 for Details):

Antcin K (25R, 25S): ¹H NMR (400 MHz, pyridine-d₅) δ: 2.10, 3.16 (2H, m, H-1), 1.95, 2.77 (2H, m, H-2), 4.09 (1H, brs, H-3), 4.64 (1H, t, J=8.2 Hz, H-7), 2.46, 2.99 (2H, d, J=13.4 Hz, H-12), 0.92 (3H, s, H-18), 2.08 (3H, s, H-19), 0.91 (3H, d, J=6.6 Hz, H-21), 1.52 (3H, d, J=7.0 Hz, H-27), 5.09 (1H, s, H-28a), 5.23 (1H, s, H-28b), 1.75 (3H, s, H-29). ¹³C NMR (100 MHz, pyridine-d₅) δ: 30.0 (C-1), 27.1 (C-2), 211.7 (C-3), 75.1 (C-4), 43.9 (C-5), 30.5 (C-6), 71.2 (C-7), 144.4 (C-8), 154.6 (C-9), 39.1 (C-10), 201.8 (C-11), 59.2 (C-12), 48.3 (C-13), 54.1 (C-14), 25.8 (C-15), 28.6 (C-16), 55.2 (C-17), 12.9 (C-18), 21.3 (C-19), 36.6 (C-20), 19.0 (C-21), 34.8 (C-22), 32.3 (C-23), 150.7 (C-24), 46.9 (C-25), 177.3 (C-26), 17.4 (C-27), 110.8 (C-28), 28.4 (C-29).

Antcin C (25R, 25S): ¹H NMR (400 MHz, pyridine-d₅) δ: 1.25, 2.90 (2H, m, H-1), 2.38 (2H, m, H-2), 4.53 (1H, t, J=8.6 Hz, H-7), 2.47 (1H, d, J=13.8 Hz, H-12a), 3.00 (1H, d, J=13.8 Hz, H-12b), 0.90 (3H, s, H-18), 1.61 (3H, s, H-19), 0.92 (3H, d, J=5.1 Hz, H-21), 1.53 (3H, d, J=7.0 Hz, H-27), 5.10 (1H, s, H-28a), 5.25 (1H, s, H-28b), 1.13 (3H, d, J=6.5 Hz, H-29). ¹³C NMR (100 MHz, pyridine-d₅) δ: 36.6 (C-1), 38.5 (C-2), 211.8 (C-3), 44.5 (C-4), 49.0 (C-5), 33.9 (C-6), 69.7 (C-7), 141.3 (C-8), 156.2 (C-9), 37.8 (C-10), 201.7 (C-11), 58.9 (C-12), 48.3 (C-13), 54.0 (C-14), 25.8 (C-15), 28.6 (C-16), 55.1 (C-17), 12.9 (C-18), 18.1 (C-19), 36.5 (C-20), 19.0 (C-21), 34.8 (C-22), 32.3 (C-23), 150.3 (C-24), 46.9 (C-25), 176.8 (C-26), 17.4 (C-27), 110.9 (C-28), 12.3 (C-29).

To determine the stereochemistry of the C-25, a Mosher ester reaction was performed using (R)- and (S)-1-(9-anthryl)-2,2,2-trifluoroethanol reagents. Firstly, 25S-Antcin C (14.67 mg, approximately 0.031 mmol), (R)-1-(9-anthryl)-2,2,2-trifluoroethanol (8.56 mg, approximately 0.031 mmol), EDCI (17.83 mg, approximately 0.093 mmol), Et₃N (8.7 μL, approximately 0.031 mmol), and DMAP (5.74 mg, approximately 0.047 mmol) were dissolved in 1 mL of deuterated chloroform. The mixture was sonicated for 20 minutes and then allowed to stand for 1 day. After the reaction was complete, the mixture was concentrated under reduced pressure, extracted with chloroform-water, and the organic layer was evaporated to obtain the product. Similarly, the 25R ester of 25S-Antcin C and the 25R S ester of 25R-Antcin C were prepared using the same procedure. The stereochemistry of the C-25 of the compound was inferred by comparing the ¹H NMR data of the resulting esters.

The NMR data of 25R S-Antcin C were compared with those of the products obtained from the Mosher reaction to determine the stereochemistry of the C-25 in the isolated 25R S-Antcin C. The specific ¹H NMR data are detailed in Table 1.

TABLE 1
Partial ¹H NMR data of Mosher ester of 25R/S-Antcin
C (400M, Pyridine-d₅, δ in ppm, J in Hz)

| | 25S-Antcin C | | 25R-Antcin C | |
|---|---|---|---|---|
| H | R^{a} | S^{b} | R^{a} | S^{b} |
| 18 | 0.80 s | 0.87 s | 0.89 s | 0.79 s |
| 21 | 0.54 d (6.0) | 0.79 d (4.2) | 0.82 d (6.0) | 0.56 d (6.0) |
| 27 | 1.37 d (7.2) | 1.34 d (7.2) | 1.32 d (7.2) | 1.37 d (7.2) |
| 28 | 4.90 s | 5.11 s | 5.10 s | 4.89 s |
| | 5.03 s | 5.15 s | 5.16 s | 5.00 s |

^{a}(R)-1-(9-anthracyl)-2,2,2-trifluoroethanol ester;
^{b}(S)-1-(9-anthracyl)-2,2,2-trifluoroethanol ester.

### Example 2: Synthesis of AK-GLU

AK-GLU is a synthetic product obtained by glycosylation of Antcin K, which is naturally occurring in Antrodia camphorata. The synthesis method is illustrated as follows.

Weigh appropriate amounts of 25R-Antcin K (9.5 mg, 0.02 mM) and 25S-Antcin K (9.9 mg, 0.02 mM) and dissolve them in 120 mL of 50 mM NaH₂PO₄—Na₂HPO₄ buffer (pH 8.0). Add two equivalents of UDP-Glc (25.5 mg, 0.04 mM) and YjiC1 enzyme solution (240 μg) and react at 37° C. with shaking at 200 rpm for 8 hours. Add methanol (2× volume) to terminate the reaction, extract with ethyl acetate (2× volume) three times, concentrate the ethyl acetate layer under reduced pressure, and re-dissolve in methanol.

Use semi-preparative liquid chromatography with a YMC Pack ODS-A column (10×250 mm, 5 μm) under the following conditions: 0-35 min, 15%-80% B; 35-45 min, 80%-100% B. Detection wavelength: 254 nm, flow rate: 2 mL/min. This yielded 25R-Antcin K-7-O-glucoside (11.1 mg, 85% yield, white solid) and 25S-Antcin K-7-O-glucoside (11.3 mg, 87% yield, white solid). The structures were confirmed by NMR and HRESIMS.

25R-Antcin K-7-O-glucoside, yield: 85%, 11.1 mg. HRESIMS: m/z 649.3582 ([M−H]⁻, C₃₅H₅₃O₁₁ Calculated value: 649.3588). ¹H NMR (400 MHz, pyridine-d₅): δ: 1.25, 3.13 (2H, m, H-1), 1.97, 2.78 (2H, m, H-2), 4.09 (1H, brs, H-3), 2.22 (1H, m, H-5), 4.63 (1H, t, J=8.4 Hz, H-7), 2.44 (1H, d, J=13.2 Hz, H-12a), 2.92 (1H, d, J=13.3 Hz, H-12b), 2.72 (1H, m, H-14), 0.82 (3H, s, H-18), 2.08 (3H, s, H-19), 0.85 (3H, d, J=6.0 Hz, H-21), 3.47 (3H, q, J=6.9 Hz, H-25), 1.51 (3H, d, J=7.0 Hz, H-27), 5.06 (1H, overlap, H-28a), 5.23 (1H, s, H-28b), 1.84 (3H, s, H-29), 5.05 (1H, overlap, H-1′), 3.99 (1H, m, H-2′), 4.04 (1H, m, H-3′), 4.19 (1H, m, H-4′), 4.26 (1H, m, H-5′), 4.30, 4.55 (2H, m, H-6′). ¹³C NMR (100 MHz, pyridine-d₅) δ: 30.0 (C-1), 27.0 (C-2), 74.9 (C-3), 74.4 (C-4), 43.7 (C-5), 29.6 (C-6), 79.7 (C-7), 151.4 (C-8), 146.7 (C-9), 38.6 (C-10), 201.9 (C-11), 59.0 (C-12), 48.5 (C-13), 54.3 (C-14), 25.1 (C-15), 28.5 (C-16), 55.3 (C-17), 12.7 (C-18), 21.2 (C-19), 36.6 (C-20), 18.9 (C-21), 34.9 (C-22), 32.1 (C-23), 150.8 (C-24), 47.1 (C-25), 177.2 (C-26), 17.5 (C-27), 110.8 (C-28), 28.3 (C-29), 105.6 (C-1′), 76.0 (C-2′), 78.5 (C-3′), 72.8 (C-4′), 79.0 (C-5′), 63.7 (C-6′).

25S-Antcin K-7-O-glucoside, yield: 87%, 11.3 mg. HRESIMS: m/z 649.3594 ([M−H]⁻, C₃₅H₅₃O₁₁ Calculated value: 649.3588). ¹H NMR (400 MHz, pyridine-d₅): δ:1.25, 3.13 (2H, m, H-1), 1.97, 2.78 (2H, m, H-2), 4.09 (1H, brs, H-3), 2.22 (1H, m, H-5), 4.63 (1H, t, J=8.4 Hz, H-7), 2.44 (1H, d, J=13.2 Hz, H-12a), 2.92 (1H, d, J=13.3 Hz, H-12b), 2.72 (1H, m, H-14), 0.82 (3H, s, H-18), 2.08 (3H, s, H-19), 0.85 (3H, d, J=6.0 Hz, H-21), 3.47 (3H, q, J=6.9 Hz, H-25), 1.51 (3H, d, J=7.0 Hz, H-27), 5.06 (1H, overlap, H-28a), 5.23 (1H, s, H-28b), 1.84 (3H, s, H-29), 5.05 (1H, overlap, H-1′), 3.99 (1H, m, H-2′), 4.04 (1H, m, H-3′), 4.19 (1H, m, H-4′), 4.26 (1H, m, H-5′), 4.30, 4.55 (2H, m, H-6′). ¹³C NMR (100 MHz, pyridine-d₅) δ: 30.0 (C-1), 27.0 (C-2), 74.9 (C-3), 74.4 (C-4), 43.7 (C-5), 29.6 (C-6), 79.7 (C-7), 151.4 (C-8), 146.7 (C-9), 38.6 (C-10), 201.9 (C-11), 59.0 (C-12), 48.5 (C-13), 54.3 (C-14), 25.1 (C-15), 28.5 (C-16), 55.3 (C-17), 12.7 (C-18), 21.2 (C-19), 36.6 (C-20), 18.9 (C-21), 34.9 (C-22), 32.1 (C-23), 150.8 (C-24), 47.1 (C-25), 177.2 (C-26), 17.5 (C-27), 110.8 (C-28), 28.3 (C-29), 105.6 (C-1′), 76.0 (C-2′), 78.5 (C-3′), 72.8 (C-4′), 79.0 (C-5′), 63.7 (C-6′).

### Example 3: Study on the Extraction Method of Antrodia camphorata

Compared to the ethanol extraction used in the prior art and in Example 1, the extraction method using a methanol-water solution results in a higher content of Antcin K and Antcin C in the extract, with no DEA present. This indicates a better enrichment of these two active components, Antcin K and Antcin C. The following experiments validate this conclusion.

The Antrodia camphorata was crushed and subjected to ultrasonic extraction with 10 times its volume of 95% ethanol and 50% methanol for 30 minutes, respectively. After extraction, the solvent was recovered by concentrating under reduced pressure to obtain the dry extracts. The contents of Antcin K, Antcin C, and DEA in the dry extracts were measured by HPLC. Standard solutions of Antcin K, Antcin C, and DEA were continuously diluted and injected into the HPLC system to create peak area-concentration curves. The HPLC conditions are:
Instrument: Agilent 1260 High-Performance Liquid Chromatograph;
Chromatographic Column: YMC-C18 Column (5 μm, 4.6×250 mm), Zorbax SB-C18 Guard Column (5 μm, 4.6×12.5 mm);
Mobile Phase: Acetonitrile (A)—0.1% Formic Acid (B);
Elution Program: 0-15 min, 40-47% A; 15-45 min, 47-67% A; 45-50 min, 67-100% A; 50-60 min, 100% A.
Detection Wavelength: 254 nm.

The HPLC chromatograms of each extract and standard are shown in FIG. 5, and the results are presented in Table 2.

TABLE 2
Determination of components in extract

| Extract | Antcin K | Antcin C | DEA |
|---|---|---|---|
| 95% ethanol | 10.20% | 0.85% | 1.12% |
| 50% methanol | 15.56% | 8.78% | 0% |

### Example 4: Effect on FGF21 Gene Expression in the Liver of MCD Mice

Tranzol (TransGen, Beijing), chloroform, ethanol, isopropanol (Beijing Chemical Works, Beijing), RNase-free double-distilled water, reverse transcription reagent kit (TransGen, Beijing), SYBR Green I fluorescence dye quantitative PCR reagents (TransGen, Beijing).

Four-week-old male C57BL/6J mice were purchased from the Experimental Animal Center, Peking University Health Science Center. The mice were randomly divided into groups, with 10 mice per group. Methionine choline-deficient diet (MCD) was used to induce non-alcoholic steatohepatitis (NASH), forming the MCD model mice. The model mice were treated with an oral administration of drugs for 4 weeks. After the final treatment, the mice were fasted overnight, and then sacrificed. Serum and liver tissues were stored at −80° C., which could be compared with untreated MCD mice (“MCD”) and normal mice (“Nor” or “Normal”). The dosing amounts for each group were as follows: low-dose Antcin C (“AC-L”): 20 mg/kg; high-dose Antcin C (“AC-H”): 40 mg/kg; DEA: 20 mg/kg; AK-GLU: 26.6 mg/kg.

Total RNA Extraction: Liver tissue samples from the mice (10-20 mg) were cut into pieces and added to 1 ml of Tranzol. The mixture was placed on ice and homogenized, then transferred to an RNase-free and DNase-free EP tube. To each tube, 0.2 ml of chloroform was added, and the mixture was vigorously shaken for 15 seconds. After incubation at low temperature for 3 minutes, the mixture was centrifuged at 12000 rpm for 15 minutes at 4° C. The aqueous upper phase was transferred to a new EP tube, and an equal volume of isopropanol was added. The mixture was inverted to mix and incubated at low temperature for 20-30 minutes. After centrifugation at 12000 rpm for 15 minutes at 4° C., a white, transparent gel-like substance was visible at the bottom of the tube. The supernatant was discarded, and 1 ml of 75% ethanol was added. The tube was vortexed and centrifuged at 12000 rpm for 5 minutes at 4° C. The supernatant was discarded, and the pellet was air-dried at room temperature. Finally, 20-50 μL of RNase-free double-distilled water was added, and the RNA was dissolved by gently pipetting. RNA concentration was measured using a spectrophotometer with an A260/280 ratio.

Reverse Transcription: RNA concentration was adjusted to a consistent level. The PCR system included 2 μg of RNA and RNase-free double-distilled water adjusted to 15 μL, 4 μL of All-in-one Mix, and 1 μL of gDNA remover. The PCR program is detailed in Table 3.

TABLE 3
The PCR program of reverse transcription

| | | |
|---|---|---|
| 25° C. | 10 | min |
| 45° C. | 15 | min |
| 85° C. | 5 | s |

| | |
|---|---|
| 4° C. | ∞ |

Real-time Quantitative PCR: The qPCR primer information is shown in Table 4. After preparing the reaction system according to Table 5, perform the qPCR experiment following the procedure outlined in Table 6, and then collect and process the data.

TABLE 4
qPCR primer information

| Primers | |
|---|---|
| (mouse) | Sequence |
| β-Actin | F: 5′-GTGACGTTGACATCCGTAAAGA-3′ |
| | R: 5′-GCCGGACTCATCGTACTCC-3′ |
| Fgf21 | F: 5′-TACAATGTGTACCAGTCTGAAG-3′ |
| | R: 5′-ACAGCCCTAGATTCAGGA-3′ |

TABLE 5
qPCR reaction system

| | volume (μL) | total | |
|---|---|---|---|
| cDNA template | 1 | 10-100 | ng |
| 2×SuperMix | 10 | 1× | |
| Forward Primer | 0.4 | 0.2 | μM |
| Reverse Primer | 0.4 | 0.2 | μM |

| | | |
|---|---|---|
| Nuclease-free water | Add to 20 | — |

TABLE 6
qPCR reaction program

| | | |
|---|---|---|
| Temperature ° C. | Time | Cycles |

| | | | |
|---|---|---|---|
| 94 | 30 | s | 1 |
| 94 | 5 | s | 40 |
| 56 | 15 | s | |
| 72 | 10 | s | |
| 95 | 1 | min | 1 |
| 55 | 30 | s | |
| 95 | 30 | s | |

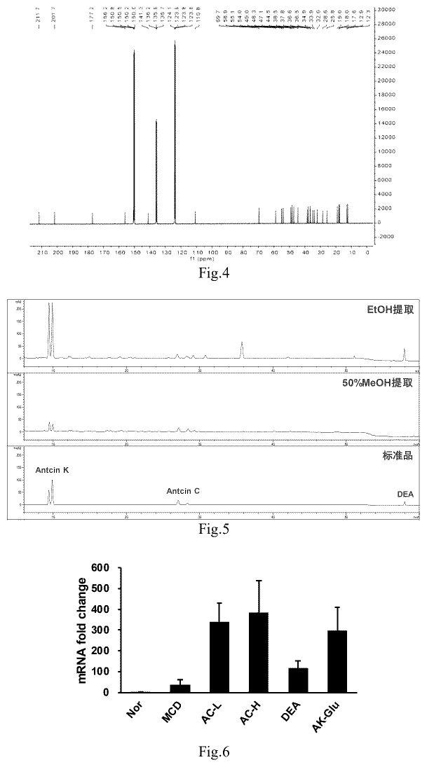

As shown in FIG. 6, compared to the DEA component reported in the literature, both Antcin C and AK-GLU significantly activated the gene expression levels of FGF21 in vivo. Antcin C and AK-GLU can increase the FGF21 expression levels in the liver of MCD mice to more than 300 times than that of normal mice.

### Example 5: Effect on FGF21 Protein Expression in the Liver of MCD Mice

The experimental materials used in this example include: methionine choline deficient diet (Research Diets, USA), RIPA lysis buffer, BSA protein assay kit (Beyotime, Shanghai), FGF21 antibody, GAPDH antibody (Bioss, Beijing), goat anti-mouse secondary antibody, and goat anti-rabbit secondary antibody (Easybio, Beijing).

The modeling and procedures for MCD mice are as described in Example 4. Comparisons were made between the treated groups and untreated MCD mice (“MCD”) and normal mice (“Normal”). The dosing for each treatment group was as follows: positive drug obeticholic acid (“OCA”): 10 mg/kg; Antcin K (“AK”): 20 mg/kg; low-dose Antcin C (“AC-L”): 20 mg/kg; high-dose Antcin C (“AC-H”): 40 mg/kg; DEA: 20 mg/kg.

Liver tissue (15-20 mg) was homogenized in RIPA lysis buffer on ice, and then centrifuged at 13,000 rpm for 30 minutes at 4° C. The middle layer liquid was transferred to a new centrifuge tube and the protein concentration was measured using BCA method. For each sample, 20 μg of protein was separated by SDS-PAGE and then transferred to a PDVF membrane using Bio-Rad's standard wet transfer system at 200 mA for 60 minutes. Subsequently, the membrane was cut according to protein molecular weight and blocked with 0.5% BSA at room temperature for 60 minutes. The primary antibody was incubated at 4° C. for 14 hours, followed by washing with TBST at room temperature 3 times for 5 minutes each. The secondary antibody was incubated at room temperature for 1 hour, followed by washing with TBST at room temperature 3 times for 10 minutes each. ECL reagent was used for detection.

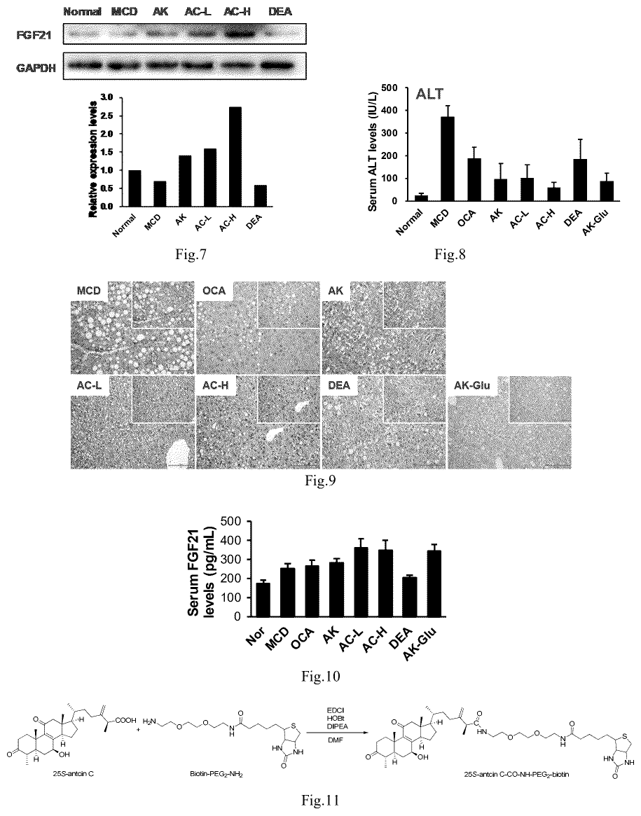

As shown in FIG. 7, compared to DEA reported in the literature, Antcin K and various concentrations of Antcin C significantly upregulated the FGF21 protein expression levels.

### Example 6: Therapeutic Effect on Non-Alcoholic Steatohepatitis (NASH) in MCD Mice

The MCD mouse model and drug administration method were the same as in Example 4. After the final treatment, the mice were sacrificed, and the liver tissue samples were fixed overnight in 4% paraformaldehyde, embedded in paraffin, and sectioned. The sections were stained with hematoxylin and eosin (H&E) according to the following procedure: immerse the sections in the following solutions sequentially: xylene (I) for 15 minutes, xylene (II) for 15 minutes, 50% xylene-absolute ethanol for 2 minutes, absolute ethanol (I) for 5 minutes, absolute ethanol (II) for 5 minutes, 80% ethanol for 5 minutes, distilled water for 5 minutes, hematoxylin staining solution for 5 minutes, rinse with running water for 5 minutes, 1% hydrochloric acid ethanol for 30 seconds, rinse with water for 30 seconds, wash with distilled water for 5 seconds, 0.5% eosin staining solution for 2 minutes, rinse with distilled water for 30 seconds, 80% ethanol for 30 seconds, 95% ethanol (I) for 1 minute, 95% ethanol (II) for 1 minute, absolute ethanol (I) for 3 minutes, absolute ethanol (II) for 3 minutes, xylene (I) for 3 minutes, xylene (II) for 3 minutes, and mount with neutral resin.

The treated groups were compared with untreated MCD mice (“MCD”) and normal mice (“Nor”). The drug administration doses were as follows: positive control obeticholic acid (OCA): 10 mg/kg; Antcin K (AK): 20 mg/kg; low-dose Antcin C (AC-L): 20 mg/kg; high-dose Antcin C (AC-H): 40 mg/kg; DEA: 20 mg/kg; AK-GLU: 26.6 mg/kg.

After the final treatment, the mice were fasted overnight, sacrificed, and blood was collected from the eye vein. The whole blood was allowed to stand for 2 hours, centrifuged at 6000 rpm for 10 minutes, and the serum was obtained. The serum ALT levels were measured using an ALT kit (Bioss, Beijing), as shown in FIG. 8. Antcin K, Antcin C, and AK-Glu significantly reduced ALT levels, demonstrating their good liver-protective activity, with effects significantly better than the positive control obeticholic acid and the reported DEA.

Additionally, FIG. 9 showed the pathological staining results. After 4 weeks, the MCD model mice exhibited significant steatosis, ballooning and other degenerative changes in the liver, and lesions were diffuse, with NAS scores ≥4, which was diagnosed as NASH lesions. Treatment with Antcin K, Antcin C, and AK-GLU significantly reduced steatosis and ballooning in the liver, alleviating the lesions, with effects significantly better than the positive control obeticholic acid and the reported DEA.

### Example 7: Effect on Serum FGF21 Protein Expression in MCD Mice

The modeling and treatment of MCD mice were conducted as described in Example 4. The treated groups were compared with untreated MCD mice (“MCD”) and normal mice (“Nor”). The dosages for each treatment group were as follows: positive control obeticholic acid (“OCA”): 10 mg/kg; Antcin K (“AK”): 20 mg/kg; low-dose Antcin C (“AC-L”): 20 mg/kg; high-dose Antcin C (“AC-H”): 40 mg/kg; DEA: 20 mg/kg; AK-GLU: 26.6 mg/kg.

The serum sampling method from mice was the same as in Example 6. The FGF21 levels were tested using an ELISA kit (Beyotime), with the following procedure: The microplate was equilibrated at room temperature for 20 minutes. Then, 50 μL of serum samples or different concentrations of standards were added to each well. Next, 100 μL of HRP-conjugated detection antibody was added, and the plate was covered with a sealing film and incubated at 37° C. for 60 minutes. The liquid was discarded, and the plate was dried with absorbent paper. Each well was then washed with 350 μL of washing buffer, left for 1 minute, and the liquid was discarded and dried with absorbent paper. This washing step was repeated 5 times. Then, 50 μL of Substrate A and 50 μL of Substrate B were added to each well and incubated at 37° C. in the dark for 15 minutes. After that, 50 μL of stop solution was added to each well, and the absorbance at 450 nm was measured within 15 minutes. A standard curve was plotted with the absorbance of the standards on the vertical axis and the standard concentrations on the horizontal axis. The serum sample absorbance values were used to determine the FGF21 protein concentrations based on this standard curve.

As shown in FIG. 10, compared to the component DEA reported in the literature, Antcin K, AK-GLU, and different concentrations of Antcin C significantly upregulated the serum FGF21 protein levels in mice.

### Example 8: Synthesis and Structural Identification of the Molecular Probe 25S-Antcin C—CO—NH-PEG-Biotin (ACS-Biotin)

The experimental materials used in this example include: 25S-Antcin C (obtained from Example 1), EDCI (1-ethyl-(3-dimethylaminopropyl) carbodiimide hydrochloride, Bidepharm, Shanghai), HOBt (1-hydroxybenzotriazole, Bidepharm, Shanghai), DIPEA (N,N-diisopropylethylamine, Energy chemical, Beijing), DMF (N,N-dimethylformamide, Energy chemical, Beijing), Biotin-PEG₂-NH₂ (N-(2-(2-(2-aminoethoxy)ethoxy)ethyl))-5-((3aS,4S,6aR)-2-oxotetrahydro-1H-thieno[3,4-d]imidazol-4-yl)pentanamide, Bidepharm, Shanghai), dichloromethane (J&K Scientific, Beijing), ammonium chloride (TGREAG, Beijing), sodium sulfate (Leyan, Beijing), chromatographic methanol (ThermoFisher, Beijing), methanol (TGREAG, Beijing), preparative-grade acetonitrile (J&K Scientific, Beijing), trifluoroacetic acid (Energy chemical, Beijing).

According to the chemical reaction shown in FIG. 11, 25S-Antcin C (20.00 mg, 0.0425 mmol), EDCI (10.61 mg, 0.5525 mmol), HOBt (7.46 mg, 0.05525 mmol), and DIPEA (47.28 μL, 0.2677 mmol) were dissolved in anhydrous DMF (1 mL). Then, Biotin-PEG₂-NH₂ (19.07 mg, 0.051 mmol) dissolved in anhydrous DMF (0.5 mL) was added. The reaction was stirred at room temperature for 10 hours. After the reaction, the system was diluted with dichloromethane and sequentially washed with saturated ammonium chloride solution, water, and saturated saline. The organic layer was dried over anhydrous sodium sulfate, filtered, and concentrated. The residue was redissolved in 1-2 mL of chromatographic methanol.

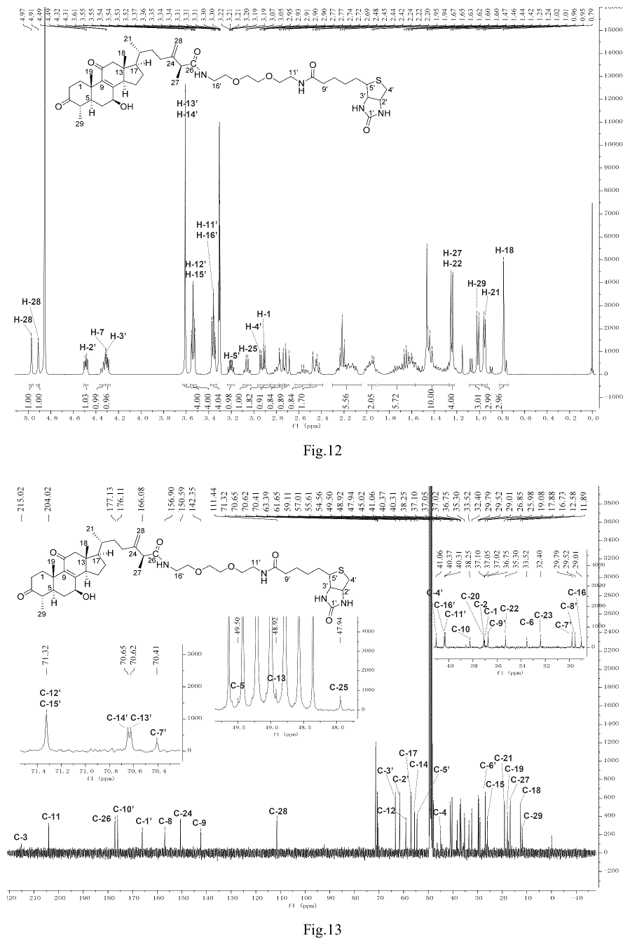

The product was purified using semi-preparative liquid chromatography with a YMC Pack ODS-A column (10×250 mm, 5 μm). Chromatographic conditions: 0-70 min, 37% B (where B is preparative-grade acetonitrile and A is 0.03% trifluoroacetic acid in water); detection wavelength: 254 nm; flow rate: 2 mL/min. The compound 25S-Antcin C—CO—NH-PEG₂-biotin (19.33 mg, yield 65%, white solid) was obtained and its structure was confirmed by NMR. The NMR spectra are shown in FIGS. 12-13.

25S-Antcin C—CO—NH-PEG₂-biotin, yield: 55%, 19.33 mg. HRESIMS: m/z 827.49871 ([M+H]⁺, C₃₅H₅₃O₁₁ calculated value: 826.49088). ¹H NMR (400 MHz, pyridine-d₅): δ:2.90, 1.45 (2H, H-1), 2.55, 2.22 (2H, H-2), 2.45 (1H, H-4), 2.41 (1H, H-5), 2.18, 1.58 (2H, H-6), 4.34 (1H, H-7), 2.46, 2.75 (2H, H-12), 2.78 (1H, H-14), 2.14 (2H, H-15), 1.96 (2H, H-16), 1.46 (1H, H-17), 0.79 (3H, H-18), 1.47 (3H, H-19), 1.46 (1H, H-20), 0.95 (3H, H-21), 1.60, 1.24 (2H, H-22), 2.12, 1.99 (2H, H-23), 3.06 (1H, H-25), 1.25 (3H, H-27), 4.97, 4.91 (2H, H-28), 1.01 (3H, H-29), 4.49 (1H, H-2′), 4.30 (1H, H-3′), 2.92, 2.71 (2H, H-4′), 3.20 (1H, H-5′), 1.66 (2H, H-6′), 1.44 (2H, H-7′), 1.73 (2H, H-8′), 2.22 (2H, H-9′), 3.36 (2H, H-11′), 3.54 (2H, H-12′), 3.61 (2H, H-13′), 3.61 (2H, H-14′), 3.54 (2H, H-15′), 3.36 (2H, H-16′). ¹³C NMR (100 MHz, pyridine-d₅) δ: 37.0 (C-1), 37.0 (C-2), 215.0 (C-3), 45.0 (C-4), 49.5 (C-5), 33.5 (C-6), 70.4 (C-7), 156.9 (C-8), 142.3 (C-9), 38.2 (C-10), 204.0 (C-11), 59.1 (C-12), 48.9 (C-13), 54.6 (C-14), 26.0 (C-15), 29.0 (C-16), 55.6 (C-17), 12.6 (C-18), 17.9 (C-19), 37.1 (C-20), 19.1 (C-21), 35.3 (C-22), 32.4 (C-23), 150.6 (C-24), 47.9 (C-25), 177.1 (C-26), 16.7 (C-27), 111.4 (C-28), 11.9 (C-29), 166.1 (C-1′), 61.6 (C-2′), 63.4 (C-3′), 41.1 (C-4′), 57.0 (C-5′), 26.9 (C-6′), 27.8 (7′), 29.5 (C-8′), 36.7 (C-9′), 176.1 (C-10′), 40.3 (C-11′), 71.3 (C-12′), 70.6 (C-13′), 70.6 (C-14′), 71.3 (C-15′), 40.4 (C-16′).

### Example 9: Protein Fishing to Identify the Target RDH10 Bound by 25S-Antcin C (ACS)

### Avidin Microspheres (Sigma, USA), Other Reagents (Beijing Chemical Works, Beijing)

### Protein Fishing Steps:

1. Preparation of Liver Protein: Take approximately 50 mg of liver tissue from C57BL/6J mice and add 1 mL of RIPA lysis buffer. Lyse the proteins on ice, then centrifuge at 12,000 rpm for 30 minutes at 4° C. Collect the supernatant, measure the protein concentration using BCA method, and dilute to 1 mg/mL with PBS.
2. Sample Preparation: Prepare new 1.5 mL EP tubes and divide them into control group, drug group, and competitive inhibition group. Add 100 μL of PBS solution containing 40 μM Biotin to the control group, 100 μL of PBS solution containing 40 μM ACS-biotin (prepared in Example 1) to the drug group, and 100 μL of PBS solution containing 40 μM ACS-biotin and 400 μM ACS to the competitive inhibition group. Then, add 100 μL of liver protein lysate (1 mg/mL) to each tube and incubate on a shaking incubator at 4° C. for 2 hours.
3. Washing Streptavidin Beads: Add 100 μL of streptavidin beads to new 1.5 mL EP tubes and centrifuge at 2,700 rpm for 1 minute. Discard the supernatant and wash the beads with 100 μL of PBS three times.
4. Binding and Washing: Add the solutions from step 2 to the washed streptavidin beads. Incubate on a shaking incubator at 4° C. for 2 hours, then centrifuge at 2,700 rpm for 1 minute. Discard the supernatant and wash the beads with 100 μL of PBS three times.
5. Protein Elution: Add 50 μL of PBS to the beads, mix well, and then add 5×SDS-PAGE sample buffer. Boil for 5 minutes and centrifuge at 13,000 rpm for 5 minutes. Store the supernatant at −20° C. for SDS-PAGE gel electrophoresis.
6. Protein Detection: Stain with Coomassie Brilliant Blue and cut the gel for proteomics analysis.

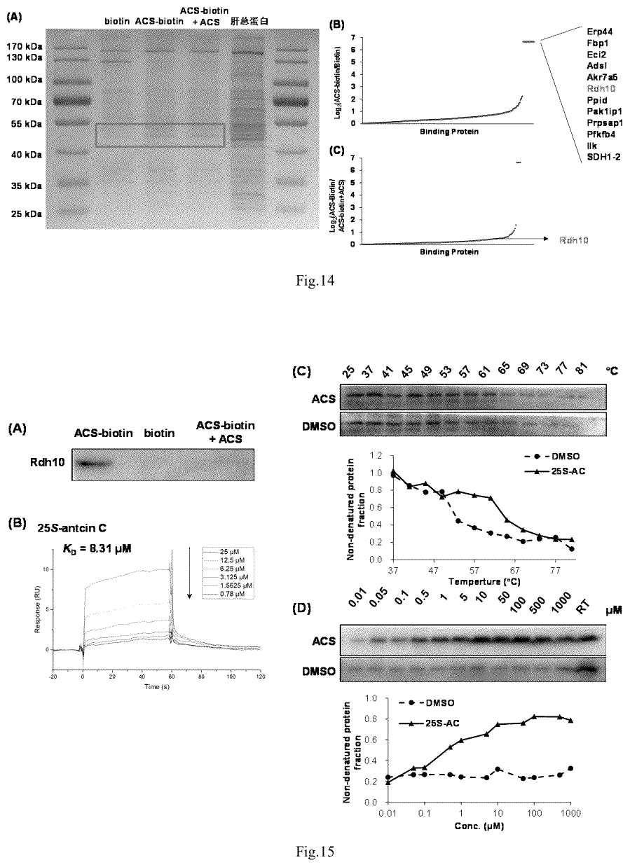

As shown in FIG. 14A, compared to the control group and the competitive inhibition group, the drug group exhibited a significantly darker protein band between 40-50 kDa. This band may contain target proteins with high binding affinity for the compound 25S-Antcin C. Cut this protein band and perform proteomics analysis on the sample. According to the proteomics results shown in FIG. 14B, compared to the control group, there were 342 proteins with log₂ (ACS-biotin/Biotin) >0 in the drug group. Among them, 23 proteins showed the greatest difference (>6), including RDH10, a molecule highly associated with reduced lipid deposition. As illustrated in FIG. 14C, compared to the competitive inhibition group, the log₂ (ACS-biotin/ACS-biotin+ACS) for RDH10 protein in the drug group is >0. This indicates that the addition of 25S-Antcin C as a competitive inhibitor reduces the binding of the molecular probe to the RDH10 protein.

### Example 10: RDH10 as a Direct Target of 25S-Antcin C Verified by Western Blot, SPR and CETSA

The experimental materials used in this embodiment include: RIPA lysis buffer, BSA protein assay kit (Beyotime, Shanghai), RDH10 antibody, GAPDH antibody (Bioss, Beijing), goat anti-mouse secondary antibody, and goat anti-rabbit secondary antibody (Easybio, Beijing).

### Western Blot Steps:

1. Tissue Collection: For each mouse, cut approximately 100 mg of liver tissue from the same area of the liver, wash with pre-chilled PBS, and place into a pre-chilled 1.5 mL EP tube.
2. Preparation of Protein Lysis Buffer: RIPA+Phosphatase Inhibitor A (1:50)+Phosphatase Inhibitor B (1:50)+Protease Inhibitor (1:100)+0.1 M EDTA (1:100)+100 mM PMSF (1:100)
3. Protein Extraction: Add 1 mL of pre-chilled protein lysis buffer to the liver tissue, homogenize the sample with a homogenizer for 30 seconds, and place on ice.
4. Centrifugation and Protein Concentration Measurement: Centrifuge at 13,000 rpm for 30 minutes at 4° C. Transfer the supernatant to a new pre-chilled 1.5 mL EP tube, measure the protein concentration using BCA method, and dilute with PBS to 2 mg/mL.
5. Protein Denaturation: Add 5×SDS-PAGE loading buffer, mix well, and boil for 5 minutes to denature the protein. Perform SDS-PAGE electrophoresis.
6. Gel Transfer: Transfer the protein to a membrane with a constant current of 200 mA for 1 hour.
7. Blocking: Cut the transferred PVDF membrane according to the molecular weight of target protein and the position of marker. Fully immerse the PVDF membrane in blocking solution and incubate at room temperature for 1 hour.
8. Primary Antibody Incubation: Add the corresponding primary antibody diluted in 5% BSA-TBST, and incubate at 4° C. for 12-14 hours.
9. Secondary Antibody Incubation: After primary antibody incubation, wash the PVDF membrane with TBST three times, 10 minutes each. Add the corresponding secondary antibody diluted in 5% BSA-TBST, and incubate at 4° C. for 1-4 hours. After secondary antibody incubation, wash with TBST three times, 15 minutes each.
10. Exposure: Prepare ECL substrate by mixing Solution A and Solution B in a 1:1 ratio. Apply the mixture evenly to the PVDF membrane and detect using a chemiluminescence imaging system.

### Surface Plasmon Resonance Technology (SPR) Steps:

Using a Biacore T200 plasmon surface resonance instrument, RDH10 protein was immobilized on a CM5 sensor chip via an amine coupling reaction. The pH of the RDH10 protein was 4.5, and the final immobilization concentration was 50 μg/mL. The running buffer consisted of 50 mM Tris-Hcl buffer containing 150 μM NaCl, 2 mM MgCl₂, 0.05% Tween-20, and 5% DMSO. In the binding experiment, different concentrations of 25S-Antcin C (25, 12.5, 6.25, 3.12, 1.56, 0.78 μM) were dissolved in the running buffer. The flow rate was 30 μL/min, with a contact time of 60 seconds and a dissociation time of 60 seconds. Data were analyzed using Biacore software, and the kinetic analysis calculated the affinity constant (K_{D} value).

### Cellular Thermal Shift Assay (CETSA) Steps:

1. Mouse liver total protein (500 μg/mL) was incubated at room temperature for 2 hours with either 25S-Antcin C (100 μM) or an equal volume of DMSO.
2. After incubation, the 25S-Antcin C-treated group and the DMSO control group were aliquoted into 13 PCR tubes, with 30 μL per tube, and placed on ice.
3. In a PCR machine, a gradient heating program for CETSA was set up with 12 temperature points (37, 41, 45, 49, 53, 57, 61, 65, 69, 73, 77, 81° C.). The 13 samples from the 25S-Antcin C-treated group and the DMSO control group were heated at each temperature point for 3 minutes, then immediately taken out and incubated at room temperature for 3 minutes, followed by rapid cooling on ice.
4. The samples were transferred to 1.5 mL EP tubes and centrifuged at 15,000 rpm for 40 minutes at 4° C.
5. The supernatant was transferred to new EP tubes, mixed with 5×SDS-PAGE loading buffer, and boiled for 5 minutes to denature the proteins. Western Blot was then performed.

To confirm the results of the proteomics analysis, Western Blot was conducted on the protein after the steps (1) to (5) in Example 2. The grouping method for the control, drug, and competition inhibition groups was the same as in Example 2. As shown in FIG. 15A, a distinct RDH10 protein band appeared in the experimental group, whereas no significant RDH10 protein bands were observed in the control and competition inhibition groups, further confirming the proteomics analysis.

As shown in FIG. 15B, the SPR results indicate that RDH10 has a strong binding affinity with 25S-Antcin C, with a K_{D} value of 8.31 μM.

FIG. 15C showed that after incubating mouse liver total protein with 25S-Antcin C (100 μM), heating at different temperatures (37, 41, 45, 49, 53, 57, 61, 65, 69, 73, 77, 81° C.) resulted in a shift in the CETSA curve compared to the DMSO group. Notably, among 53-69° C., there was a significant increase in RDH10 protein that did not precipitate after incubation with 25S-Antcin C. On the other hand, as shown in FIG. 15D, maintaining a constant temperature of 61° C., incubation with varying concentrations of 25S-Antcin C (0.01, 0.05, 0.1, 0.5, 1, 5, 10, 50, 100, 500, 1000 μM) generated an isothermal dose-response CETSA curve (ITDRF_{CETSA}). The stability of RDH10 protein at 61° C. increased with higher concentrations of 25S-Antcin C, stabilizing after 100 μM. The CETSA curve and ITDRF_{CETSA} results indicate that 25S-Antcin C can bind to RDH10 protein, enhancing its thermal stability.

### Example 11: Cell siRNA Assay

The 1640 culture medium, KREBS buffer, and penicillin-streptomycin cell culture antibiotics used in this example were purchased from Macgene Technology Co., Ltd. (Beijing, China). Fetal bovine serum, Grade A, was purchased from Gibco (New York, USA). EGTA and collagenase IV were purchased from Huazhong Haiwei Gene Technology Co., Ltd. (Beijing, China). CaCl₂, heparin, mouse tail type I collagen, acetic acid, palmitic acid, and oleic acid were purchased from Beijing Solabio Technology Co., Ltd. (Beijing, China). Opti-MEM medium was purchased from Gibco (New York, USA). Mouse RDH10 siRNA (sc-76377), human RDH10 siRNA (sc-76376), and control siRNA (sc-37007, applicable to both human and mouse) were purchased from Santa Cruz (Dallas, USA). Lipofectamine™ RNAiMAX transfection reagent was purchased from Invitrogen (Carlsbad, USA). L02 human liver cells were obtained from the Beijing Union Cell Bank.

### Isolation of Mouse Primary Cells:

1. Prepare Reagents: Collagen I Solution: Prepare 0.02 N acetic acid using deionized water, sterile filter, and dilute collagen I to 50 μg/mL. Perfusion Solution: KREBS buffer containing 0.1 mM EGTA, sterile filter, and pre-warm to 37° C. Digestive Solution: KREBS buffer containing 2.7 mM CaCl₂) and 0.05% collagenase IV, sterile filter, and pre-warm to 37° C. Heparin Solution: Prepare 2 mg/mL heparin solution using deionized water, sterile filter.
2. Coat with Collagen: Add 2 mL of collagen I solution to each well in a 6-well plate, let it stand for 0.5-1 hour, remove the collagen solution, and expose to UV light for 5 hours.
3. Anesthetize the Mouse: After alcohol disinfection, isolate the inferior vena cava, create a pseudo-clot, insert a venous cannula below the pseudo-clot, and secure the pseudo-clot.
4. Perfuse the Mouse: Inject 1 mL of heparin into the mouse vein via the cannula, prepare to inject the perfusion solution, and simultaneously cut the portal vein. Perfuse for 3-5 minutes.
5. Digest the Liver: Inject the digestive solution for 3-6 minutes until the liver digested, then remove the gallbladder.
6. Process the Liver: Place the liver into a culture dish on ice, transfer to a sterile workbench, and repeatedly gently pipette the liver with pre-chilled 1640 medium at 4° C. After cell dissociation, pass through a 400-mesh sieve, take 20 μL of cells, add 2 μL of trypan blue staining, and observe cell viability under a microscope.
7. Centrifuge Liver Cells: Transfer the liver cells to a 50 mL centrifuge tube, centrifuge at 50×g for 2 minutes, discard the supernatant, add 25 mL of 1640 medium, centrifuge again, and repeat 3 times.
8. Resuspend Cells: Resuspend the cells in 25 mL of 1640 medium containing 10% serum, count the cells, and seed at 5×10⁵ cells/well in a 6-well plate. Change the medium after 6 hours.

Next, perform the siRNA experiment on both the isolated mouse primary liver cells and L02 liver cells as described earlier. The experimental steps were as follows:
1. Seeding Cells: Seed the cells (mouse primary liver cells or human L02 liver cells) at a density of 5×10⁵ cells per well in a 6-well plate.
2. Grouping: When the cell density reached 60%, divide the cells into the following groups: Control siRNA group, Control siRNA+Drug group, RDH10 siRNA group, and RDH10 siRNA+Drug group.
3. Prepare Transfection Solution A: In a 1.5 mL RNase-free EP tube, add 250 μL of Opti-MEM medium, then add 30 pmol of either human or mouse RDH10 siRNA, or 30 pmol of control siRNA. Mix gently.
4. Prepare Transfection Solution B: In a separate 1.5 mL RNase-free EP tube, add 250 μL of Opti-MEM medium, then add 5 μL of Lipofectamine™ RNAiMAX transfection reagent. Mix gently.
5. Combine Transfection Reagents: Add Solution A to Solution B, invert and gently mix, then let stand at room temperature for 15-20 minutes.
6. Apply Transfection Reagents: Remove the medium from the cells. For the Control siRNA+Drug group and RDH10 siRNA+Drug group, replace the medium with complete 1640 medium containing palmitic acid-oleic acid and 25S-Antcin C, without antibiotics. For the Control siRNA group and RDH10 siRNA group, replace with complete 1640 medium containing palmitic acid-oleic acid and an equal volume of DMSO, without antibiotics. Add the transfection reagent containing control siRNA to the Control siRNA group and Control siRNA+Drug group. Add the transfection reagent containing RDH10 siRNA to the RDH10 siRNA group and RDH10 siRNA+Drug group.
7. Incubation and Subsequent Detection: Incubate for 24 hours. After incubation, discard the medium, wash with PBS, and fix with 4% paraformaldehyde at room temperature for 10 minutes. Remove 4% paraformaldehyde, incubate with 60% isopropanol for 10 minutes, and then stain with Oil Red O for 20-30 minutes. Rinse quickly with 60% isopropanol several times, wash with deionized water, and mount with glycerol.

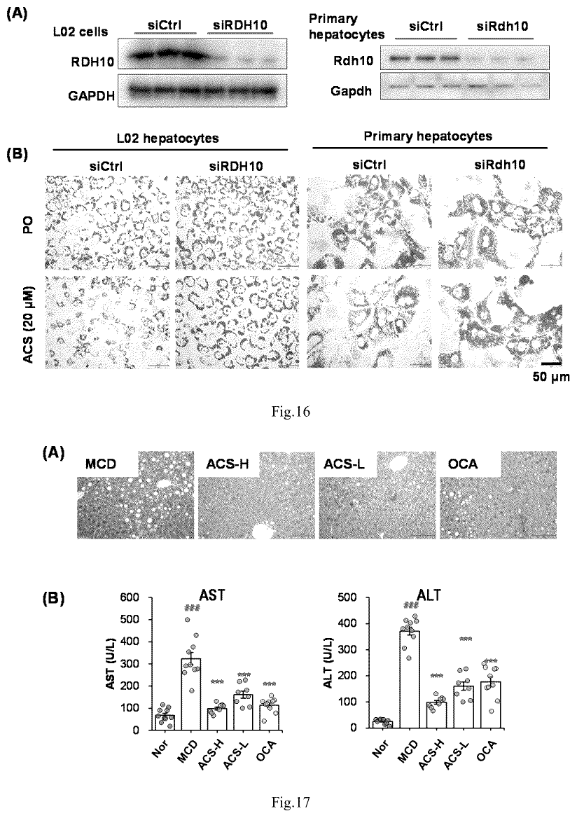

As shown in FIG. 16A, both L02 human liver cells and mouse primary liver cells with RDH10 knockdown significantly reduced the RDH10 protein expression level compared to the control siRNA group, indicating successful gene knockdown. FIG. 16B showed that oleic acid-palmitic acid induced lipid deposition in the cells. In both L02 human liver cells and mouse primary liver cells, after treatment with 25S-Antcin C (20 μM), the lipid droplets in the Control siRNA group significantly reduced in size, indicating 25S-Antcin C has a significant lipid-lowering effect. However, compared to the control siRNA group, 25S-Antcin C's effect on reducing lipid deposition in the RDH10 siRNA group was almost completely lost, with lipid size and quantity comparable to the RDH10 siRNA group. This result indicates that RDH10 knockdown decreases the lipid-lowering activity of 25S-Antcin C, suggesting that RDH10 is a target of 25S-Antcin C.

### Example 12: 25S-Antcin C Alleviating NASH in Mice

The aim of this experiment is to further validate that 25S-Antcin C can alleviate non-alcoholic steatohepatitis (NASH) in mice and its effectiveness is superior to the positive drug, based on the verification of Example 6 that Antcin C can alleviate NASH in mice.

Four-week-old male C57BL/6J mice were purchased from the Experimental Animal Center, Peking University Health Science Center. The mice were randomly divided into groups, with 10 mice per group. Methionine choline-deficient diet (MCD) was used to induce NASH, forming the MCD model mice. The model mice were treated with an oral administration of drugs for 4 weeks. After the final treatment, the mice were fasted overnight, and then sacrificed. Serum and liver tissue samples were stored at −80° C., which could be compared with untreated MCD mice (“MCD”) and normal mice (“Nor” or “Normal”). The dosing amounts for each group were as follows: low-dose 25S-Antcin C (“ACS-L”): 10 mg/kg; high-dose 25S-Antcin C (“ACS-H”): 20 mg/kg; obeticholic acid (“OCA”): 10 mg/kg.

After the final treatment, the mice were sacrificed, and the liver tissue samples were fixed overnight in 4% paraformaldehyde, embedded in paraffin, and sectioned. The sections were stained with hematoxylin and eosin (H&E) according to the following procedure: immerse the sections in the following solutions sequentially: xylene (I) for 15 minutes, xylene (II) for 15 minutes, 50% xylene-absolute ethanol for 2 minutes, absolute ethanol (I) for 5 minutes, absolute ethanol (II) for 5 minutes, 80% ethanol for 5 minutes, distilled water for 5 minutes, hematoxylin staining solution for 5 minutes, rinse with running water for 5 minutes, 1% hydrochloric acid ethanol for 30 seconds, rinse with water for 30 seconds, wash with distilled water for 5 seconds, 0.5% eosin staining solution for 2 minutes, rinse with distilled water for 30 seconds, 80% ethanol for 30 seconds, 95% ethanol (I) for 1 minute, 95% ethanol (II) for 1 minute, absolute ethanol (I) for 3 minutes, absolute ethanol (II) for 3 minutes, xylene (I) for 3 minutes, xylene (II) for 3 minutes, and mount with neutral resin. As shown in FIG. 17A, 25S-Antcin C at different concentrations significantly reduced liver steatosis and ballooning in mice, with better improvement than the positive drug obeticholic acid.

After the final treatment and overnight fasting, mice were anesthetized, and blood was collected from the tail vein. Whole blood was allowed to stand for 2 hours, centrifuged at 6000 rpm for 10 minutes, and the supernatant was used to measure serum alanine aminotransferase (ALT) and aspartate aminotransferase (AST) levels using ALT assay kit and AST assay kit (Bote Biotech, Beijing). Results are shown in FIG. 17B. The results indicate that 25S-Antcin C significantly reduced serum ALT and AST levels, demonstrating good liver protective activity and showing significantly better effects than the positive drug obeticholic acid.

1. Use of a compound of formula (I): 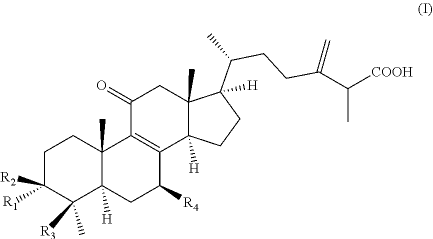
or a salt or isomer thereof in the preparation of an agonist and/or RDH10 agonist,
wherein,
R₁ and R₂ are each independently selected from the group consisting of —H, —OH and C₁₋₆ alkyl, or R₁ and R₂ together form ═O;
R₃ is selected from the group consisting of —H, —OH and C₁₋₆ alkyl; and
R₄ is selected from the group consisting of —H, —OH and a glucosyl (preferably oxy-glucosyl).

2. The use according to claim 1, wherein R₄ is oxy-glucosyl.

3. The use according to claim 1, wherein R₁ and R₂ are each independently selected from the group consisting of —OH and —H, or R₁ and R₂ together form ═O; R₃ is selected from the group consisting of —H and —OH; and R₄ is —OH or oxy-glucosyl.

4. The use according to claim 1, wherein the compound of formula (I) is Antcin K, Antcin C, AK-GLU or AC-GLU, preferably 25S-Antcin C, or a salt or isomer thereof:

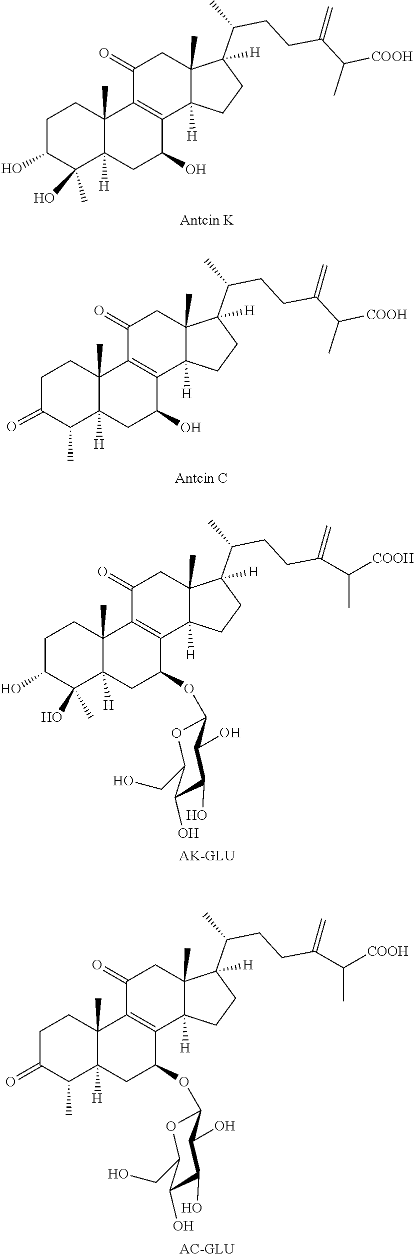

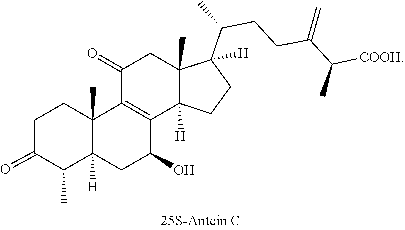 [SMILES: C=C(CC[C@@H](C)[C@H]1CC[C@H]2C3=C(C(=O)C[C@]12C)[C@@]1(C)CCC(=O)[C@@H](C)[C@@H]1C[C@@H]3O)[C@H](C)C(=O)O]

5. An Antrodia camphorata extract, wherein the extract meets one or more of the following conditions:
(1) the mass fraction of Antcin K is more than 5%, preferably more than 10%, more preferably more than 15%;
(2) the mass fraction of Antcin C is more than 3%, preferably more than 5%, more preferably more than 8%;
(3) the total mass fraction of Antcin K and Antcin C is more than 8%, preferably more than 15%, more preferably more than 20%;
(4) the mass fraction of DEA is less than 1%, preferably less than 0.5%, more preferably less than 0.1%.

6. A method of extracting Antrodia camphorata to obtain the Antrodia camphorata extract according to claim 5, comprising the following steps:
(1) crush Antrodia camphorata; and
(2) extract with an aqueous methanol solution, where the methanol concentration in the solution is 20-80%, preferably 30-70%, more preferably 40-60%.

7. A pharmaceutical composition, which contains the Antrodia camphorata extract according to claim 5.

8. Use of the Antrodia camphorata extract according to claim 5 or the pharmaceutical composition according to claim 7 in the preparation of an FGF21 agonist and/or RDH10 agonist.

9. Use of the compound of formula (I) or a salt or isomer thereof according to any one of claims 1-4, the Antrodia camphorata extract according to claim 5, or the pharmaceutical composition according to claim 7 in the preparation of a medicament for the treatment or prevention of lipid metabolism disorders (e.g., hyperlipidemia; cholesterol deposition; retinal lipemia; steatohepatitis, such as non-alcoholic steatohepatitis) or diseases related to lipid metabolism disorders (e.g., obesity; cardiovascular diseases related to lipid metabolism disorders, such as hypertension, atherosclerosis; or kidney diseases related to lipid metabolism disorders).

10. The use according to claim 9, wherein the medicament is used for the treatment or prevention of non-alcoholic steatohepatitis.
